(19) United States
(12) Patent Application Publication
(10) Pub. No.: US 2026/0283720 A1
(43) Pub. Date: Sep. 24, 2026

(54) NAVIGATION SYSTEM, MEDICAL SYSTEM, NAVIGATION METHOD, AND USE

(71) Applicant: B. Braun New Ventures GmbH, Freiburg (DE)

(72) Inventors: Simon Stehle, Freiburg (DE); Jean Stawiaski, Freiburg (DE)

(21) Appl. No.: 19/571,962

(22) Filed: Mar. 19, 2026

(30) Foreign Application Priority Data
Mar. 24, 2025 (EP) 25165673.2

(51) Int. Cl. A61B 34/30; A61B 34/20; A61B 90/00

(52) U.S. Cl. CPC A61B 34/30; A61B 2034/2057; A61B 2034/2065; A61B 2090/371; A61B 2090/3937; A61B 2090/397

(57) ABSTRACT

A navigation system for tracking a medical instrument relative to a patient includes a first navigation camera configured to capture a current position and/or orientation of the medical instrument in the visible spectrum, a second navigation camera configured to capture a current position and/or orientation of the patient in the visible spectrum, and a control unit configured to determine a current relative position and/or orientation of the medical instrument to the patient from the current position and/or orientation of the medical instrument captured by the first navigation camera and the current position and/or orientation of the patient captured by the second navigation camera. The navigation system can be incorporated into a medical system that includes a patient tracker, and can be used in a navigation method.

### CROSS-REFERENCE TO RELATED APPLICATION

This application claims priority under 35 U.S.C. § 119 to European Application No. 25165673.2, filed on Mar. 24, 2025, the content of which is incorporated by reference herein in its entirety.

### FIELD

The present disclosure relates to a navigation system for tracking a medical instrument relative to a patient. Navigation systems are widely used in computer-guided or computer-assisted surgery and help surgeons orient themselves during an operation.

### BACKGROUND

Navigation systems are used to track objects during an operation, i.e., to capture their position and/or orientation in real time (as far as possible). For example, medical instruments can be tracked to a patient to determine the current position and/or orientation of the instrument relative to an area of interest. In neurosurgery, for example, a biopsy needle can be tracked relative to a patient in order to navigate it precisely to the patient's brain tumor.

Navigation systems feature a sensor that can capture the position and/or orientation of an object, e.g., the patient or an instrument. The sensor can be a camera, for example, in which case we refer to optical tracking. Typically, navigation cameras are designed for the infrared (IR) spectrum. The object to be tracked has a tracker, in this case with IR markers that reference its position and/or orientation and can be captured by the IR navigation camera. IR markers are typically spherical and can be active or passive, i.e., they can emit IR light themselves or reflect IR light from the navigation camera.

Alternatively, the navigation camera can also be designed for tracking in the visible spectrum (wavelengths visible to humans). The object to be tracked can then be tracked, for example, based on its shape, or it can have an optical pattern, such as a barcode, which can be captured and tracked by the navigation camera in order to track the instrument.

However, optical navigation cameras often have visibility problems. However, in order to track an instrument relative to a patient, the navigation camera should always be able to see both the instrument and the patient. Normally, the patient is tracked using a patient tracker that is rigidly attached to the patient and can reference their position and/or orientation or an anatomical area of interest, e.g., a vertebra or the skull, etc. The patient tracker can also feature IR markers, for example, which can be captured by the IR navigation camera. It is understood that the navigation camera should not necessarily see the patient themselves, but at least the patient tracker and the instrument.

However, particularly during procedures on the spine or head, the navigation camera's view of a patient tracker and a medical instrument may be restricted, for example, by a surgeon or other instruments.

Typically, navigation cameras are therefore positioned at some distance from the surgical area and usually above the patient in order to provide a good overview. However, the further away the navigation camera is, the larger the trackers need to be in order to be easily recognized. However, larger trackers are more unwieldy and may interfere with the surgeon's work.

In spinal surgery in particular, it may be necessary to track several vertebrae individually, requiring a patient tracker to be attached to each vertebra. This can be problematic, especially in the case of large and unwieldy trackers, due to limited space, and is therefore disadvantageous. In addition, as the number of patient trackers increases, so does the likelihood that the line of sight to the navigation camera will be blocked for one of the patient trackers.

Furthermore, as the size of the patient tracker increases, so does the risk that medical staff may bump into it during surgery. This may cause injury to the patient or alter the patient tracker's position relative to the patient, which in turn invalidates any previous registration and renders the patient tracker unusable for navigation.

### SUMMARY

In view of the problems described above, it is therefore a task of the disclosure to avoid or at least reduce the disadvantages of the prior art. In particular, a means of tracking a medical device to a patient should be provided that causes no or minimal visual problems and is as error-free as possible, for example, by preventing accidental bumping of the patient tracker.

Advantageous embodiments are described below in the description.

The disclosed navigation system is used to track a medical instrument relative to a patient. The navigation system includes a first navigation camera configured to capture a current position and/or orientation of the medical instrument in the visible spectrum. The navigation system has a second navigation camera (separate from the first navigation camera) that is set up to capture the patient's current position and/or orientation in the visible spectrum. The navigation system has a control unit that is set up to determine the current relative position and/or orientation of the medical instrument in relation to the patient from the current position and/or orientation of the medical instrument captured by the first navigation camera and the current position and/or orientation of the patient captured by the second navigation camera.

So, a core idea of the disclosure is to use two separate cameras to track a medical instrument to a patient, with one camera tracking the medical instrument and the other camera tracking the patient. This means that it is not necessary for a camera to see both the patient and the instrument at the same time in order to determine the relative position and/or orientation of the instrument in relation to the patient. This means that the cameras are preferably arranged so that the patient is outside the field of view/direct line of sight of the camera tracking the medical instrument, or the medical instrument is outside the field of view/direct line of sight of the camera tracking the patient. The control unit can be configured, for example, to display the medical instrument in a position and/or orientation that is accurate based on the (specified) relative position and/or orientation on a radiological image of the patient, e.g., a preoperative image. In other words, the control unit is configured to determine the current relative position and/or orientation between the medical instrument and the patient based on the image of the first navigation camera containing the medical instrument, the image of the second navigation camera containing the patient, and a known transformation (i.e., relative position and/or orientation) between the two navigation cameras.

It is understood that the navigation cameras can track the patient or the instrument if they or, for example, a connected tracker are within the field of view of the respective camera.

It is understood that the position and/or orientation of the patient can also be captured indirectly via a patient tracker, meaning that the patient can also be outside the field of view of the second navigation camera. In other words, the second navigation camera is preferably set up to track a patient tracker, preferably several simultaneously and individually.

The first navigation camera can be set up to track the medical instrument itself, e.g., via its shape. However, the first navigation camera is preferably set up to track the medical instrument via an optical pattern of the medical instrument.

It should be understood that the optical pattern, and any optical pattern in this disclosure, is adapted for tracking in the visible spectrum (the spectrum visible to humans). To this end, it may have different sections or characteristics (known as features) in at least two different colors, e.g., black and white, or exhibit contrast. So these are explicitly not IR markers. The optical pattern may, for example, comprise lines (such as a barcode), squares (such as a QR code), and/or other shapes such as circles or ellipses, which may be arranged in a regular or irregular pattern.

It is understood that the first navigation camera can be set up to track several medical instruments simultaneously and individually.

The first navigation camera and the second navigation camera are designed for the visible spectrum. It goes without saying that this gives them a distinct advantage when it comes to creating optical recordings, i.e., images and/or image sequences or videos (in the visible spectrum). Preferably, the navigation system includes a display device that is configured to display images from the first and second navigation cameras. The display device can be, for example, a monitor or a head-up display.

The medical instrument is preferably a surgical instrument, i.e., designed for surgical procedures. This may be, for example, a measuring instrument such as an electrode for stereoelectroencephalography (sEEG), an imaging instrument such as an endoscope, or a manipulator for manipulating the patient, such as a (bone) drill or milling cutter. An implant may also be enclosed under the medical instrument.

The navigation system is preferably designed or configured to track the medical instrument spatially in six degrees of freedom (three translational and three rotational degrees of freedom) relative to the patient (6D tracking). However, the disclosure is not limited to this; for example, only the relative position (in three Cartesian coordinates) of the instrument to the patient can be captured or determined.

The advantages of the disclosure are that two separate navigation cameras (hereinafter also referred to as cameras) are used to track an instrument and a patient individually in order to track the instrument to the patient. This allows the two cameras to be positioned more flexibly than a single camera that has to view both the instrument and the patient at the same time, making it easier to avoid visibility problems. This also makes it possible to increase the distance between the instrument and the patient tracker while ensuring that the instrument can still be tracked in relation to the patient. As described above, both cameras are designed for the visible spectrum. On the one hand, this has the advantage that camera images can be used for image support during the procedure. On the other hand, trackers with IR markers are usually larger and more cumbersome, whereas optical patterns for tracking in the visible spectrum can be smaller and can, for example, be glued or engraved onto the instrument, thus having little or no effect on the effective size of the instrument. Another advantage is that the two cameras can have different lenses or be of different types, allowing them to be individually adapted to their respective tasks. The first navigation camera can therefore be specifically selected or adapted to track a medical instrument particularly well, whereas the second navigation camera can be specifically selected or adapted to track the patient particularly well.

Preferably, the first navigation camera and the second navigation camera are fixed in relation to each other or attached to each other. This means that the captured position data of the patient and the medical instrument can be easily exchanged or converted into a common coordinate system, as the transformation between the two cameras is known. In other words, the first navigation camera and the second navigation camera are preferably arranged immovably relative to each other.

Preferably, the first navigation camera and the second navigation camera, in particular as an end effector, are mounted together on a robotic arm. This means that they can both be moved or positioned together. Preferably, the first navigation camera and the second navigation camera are both extracorporeal.

Preferably, the first navigation camera and the second navigation camera are aligned in the same direction, i.e., they face in the same direction (as far as possible) parallel to each other. An optical axis of the first navigation camera and an optical axis of the second navigation camera are therefore, at least essentially, parallel to each other.

Preferably, the field of view of the first navigation camera overlaps with the field of view of the second navigation camera.

It is understood that the first navigation camera and the second navigation camera are preferably calibrated or calibratable individually and/or in relation to each other.

The first navigation camera and the second navigation camera differ from each other primarily in terms of their type. Preferably, the first navigation camera has optics or optical components that differ from the optics or optical components of the second navigation camera.

The first navigation camera of choice is a digital surgical microscope. The first navigation camera is therefore preferably a digital microscope (i.e., a microscope that is set up to create a digital image that can be displayed on a monitor) that is adapted for surgery or operating. The microscope can also be a hybrid microscope, which additionally has optics for direct viewing of an object located underneath it. This has the advantage that the first navigation camera can be used to display a high-resolution, enlarged view of the surgical site while simultaneously tracking the medical instrument being used in the surgical area.

Preferably, the first navigation camera is high-resolution, preferably with a small field of view, especially in comparison to the second navigation camera. Preferably, the first navigation camera has a variable focus. This allows it to be precisely focused on the instrument and/or surgical area. This allows the instrument to have an optical pattern with very fine and/or small features that can still be easily recognized by the high-resolution first navigation camera.

The second navigation camera is preferably a wide-angle camera, especially in comparison to the first navigation camera. Preferably, the second navigation camera has a larger field of view than the first navigation camera. This allows the second navigation camera to capture a larger (image) area and provide an overview image that shows a larger area than the first navigation camera. In addition, it can be used to simultaneously capture and track multiple patient trackers that are attached to the patient, e.g., to individual vertebrae.

The second navigation camera preferably has a fixed (or unchangeable) focus or depth of focus. Preferably, the second navigation camera has a large depth of field, especially in comparison to the first navigation camera. This has the advantage that the second navigation camera can be set to a sufficiently sharp and precise focus (whenever possible) without the need for time-consuming adjustments.

Preferably, the field of view of the first navigation camera lies within the field of view of the second navigation camera (at least from a certain distance or depth from the second navigation camera). In other words, the larger field of view of the second navigation camera preferably encompasses the smaller field of view of the first navigation camera (but to a certain depth). This allows the first navigation camera to capture an image of a small area of interest, such as an operating area, while the second navigation camera captures an image of the surrounding structures to provide an overview.

The first navigation camera should preferably be stereoscopic. A second navigation camera should preferably be stereoscopic.

Furthermore, the disclosure relates to a medical system with a navigation system, preferably as described above, and a patient tracker. The patient tracker is designed to be rigidly attached to a patient in order to reference their (current) position and/or orientation. The patient tracker has an optical pattern in the visible spectrum. The patient tracker is therefore adapted for tracking in the visible spectrum. The second navigation camera is set up to visually capture the optical pattern of the patient tracker. The second navigation camera is set up to track the patient via the patient tracker or the optical pattern of the patient tracker (in the visible spectrum) or to capture the patient's current position and/or orientation via the patient tracker.

It is understood that the patient tracker can also be indirectly rigidly connected or attached to the patient, for example via a head clamp. The patient tracker can therefore be designed to be rigidly attached directly to the patient and/or designed to be rigidly attached, for example, to a patient attachment device such as a head clamp, which is designed to be rigidly attached to the patient. The patient tracker preferably has a fastening section for rigidly fastening the patient tracker to the patient. The fastening section can be, for example, a screw or an adhesive surface. Rigid means that (as far as possible) there is no relative movement between the patient and the patient tracker, so that the position and/or orientation of the patient is referenced correctly, especially after initial registration.

It is understood that the medical system may have multiple (such) patient trackers and/or the second navigation camera may be configured to track multiple patient trackers, in particular simultaneously and individually, in particular to track multiple anatomical structures or areas of the patient simultaneously and individually or separately.

Preferably, the medical system includes a medical instrument, preferably as described above, wherein the medical instrument has an optical pattern in the visible spectrum and the second navigation camera is configured to visually capture the optical pattern of the medical instrument. The second navigation camera is preferably set up to track the medical instrument via its optical pattern (in the visible spectrum) or to determine the current position and/or orientation of the medical instrument.

Preferably, the optical pattern of the medical instrument is engraved, e.g., using a laser, and/or affixed. This means that it does not enlarge the medical instrument, or only very slightly.

Alternatively, the medical instrument could also have no optical pattern and/or the second navigation camera could be set up to track the medical instrument, e.g., based on its shape.

Preferably, the optical pattern of the instrument and the optical pattern of the patient tracker differ from each other in their nature.

For example, they may differ in size, color, and/or type of features (e.g., lines or circles), or in how many degrees of freedom they can specify for the instrument or patient tracker. For example, the optical pattern of the patient tracker may be designed for 5D tracking because it is rotationally symmetric, while the optical pattern of the instrument may be designed for 6D tracking.

Preferably, the optical pattern of the medical instrument is reusable and can be sterilized. For this purpose, it can be engraved, laser-engraved, and/or coated, for example. In contrast, the patient tracker may be a disposable product and/or the optical pattern of the patient tracker may not be sterilizable. The visual pattern of the patient tracker can be affixed or engraved, for example.

Preferably, the optical pattern of the medical instrument is laser-engraved or engraved, in particular with higher energy or deeper than the optical pattern of the patient tracker, in order to have lower abrasiveness.

The optical pattern of the medical instrument is preferably less reflective or has a lower reflectance than the optical pattern of the patient tracker. This can be achieved, for example, by using a darker color or a coating. This may make it more suitable for use under a surgical microscope.

Preferably, the optical pattern of the medical instrument is finer and/or smaller than the optical pattern of the patient tracker.

The optical pattern of the medical instrument is therefore preferably smaller in size than the optical pattern of the patient tracker, in particular a section that is visible from one direction. This has the advantage that it can save space and be attached directly to the instrument. The patient tracker can be larger, as it is usually not as close or direct as the instrument in the surgical area and therefore has less impact on the ergonomics for the physician.

In particular, since the first navigation camera is preferably a surgical microscope (with high resolution), the optical pattern of the patient tracker can be finer or have finer structures or features, which means that it can also be smaller overall.

Preferably, the optical pattern of the medical instrument, especially in comparison to the optical pattern of the patient tracker, is robust against (partial) occlusion. This means that the optical pattern of the medical instrument is preferably adapted so that, in the event that part of the optical pattern is obscured, e.g., by blood during an operation or by a surgeon, it still has enough exposed features that provide sufficient information to reference the position and/or orientation of the medical instrument so that the first navigation camera can (if possible) continue to track the instrument.

Preferably, the optical pattern of the medical instrument exhibits redundancies, in particular more than the optical pattern of the patient tracker.

Preferably, the medical system includes a third navigation camera (separate from the first navigation camera and the second navigation camera) for the infrared spectrum and at least one IR marker connected to the robotic arm. The third navigation camera is set up to track the IR marker in the infrared spectrum, in particular to capture the position and/or orientation of the robotic arm, preferably the first and second navigation cameras.

The IR marker can be attached, for example, to the robotic arm itself, to the end effector, or to a base of the robotic arm. This allows the position and/or orientation of the robotic arm, in particular that of the first and second navigation cameras, to be determined, if necessary, via robot kinematics, for example in order to position another medical device such as a C-arm for imaging relative to it and/or for patient registration.

It is understood that the third (IR) navigation camera is positioned or can be positioned at a distance from the robotic arm.

Furthermore, the disclosure relates to a navigation method for tracking a medical instrument relative to a patient, preferably for a navigation system and/or a medical system as described above, comprising the steps of:
capturing a current position and/or orientation of the medical instrument in the visible spectrum, preferably via an optical pattern of the medical instrument, by means of a first navigation camera;
capturing a current position and/or orientation of the patient in the visible spectrum, preferably via a patient tracker with an optical pattern, by a second, separate navigation camera; and
determining a current relative position and/or orientation of the medical instrument with respect to the patient from the current position and/or orientation of the medical instrument captured by the first navigation camera and the current position and/or orientation of the patient captured by the second navigation camera (in particular by a/the control unit).

It is understood that the first two steps mentioned above should preferably be carried out simultaneously and that the third step should preferably be carried out as quickly as possible thereafter (preferably simultaneously). In other words, the method is preferably executed in real time (if possible).

It should be noted that the method is automated and can be performed by a corresponding navigation system, for example, and therefore does not restrict the physician's activities and does not involve any surgical or therapeutic steps.

According to one aspect, the disclosure further relates to a navigation system comprising a first navigation camera, a second navigation camera, and preferably a control unit, preferably as described above, wherein the navigation system is configured to perform the steps of the method as described above.

According to another aspect, the disclosure further relates to a computer-readable storage medium having functions that cause a navigation system and/or medical system, preferably as described above, to perform the steps of the method as described above.

Furthermore, the disclosure relates to the use of a first navigation camera for capturing a current position and/or orientation of a medical instrument in the visible spectrum and a separate second navigation camera for capturing a current position and/or orientation of a patient in the visible spectrum in order to track the medical instrument relative to the patient, or in other words, to determine a current relative position and/or orientation of the medical instrument with respect to the patient.

### BRIEF DESCRIPTION OF THE DRAWINGS

The disclosure is explained in more detail below, with reference to preferred embodiments and the accompanying figures.

FIG. 1 shows a navigation system and a medical system according to the present disclosure in a preferred embodiment, exemplarily in a spinal procedure;

FIG. 2 shows the navigation system and a medical system from FIG. 1 during a neural procedure on a recumbent patient;

FIG. 3 shows a navigation system and a medical system according to the present disclosure during a procedure on a semi-seated patient; and

FIG. 4 shows a navigation method according to the present disclosure.

The figures are schematic and serve only to aid understanding of the disclosure. The features of the various embodiments can be interchanged.

### DETAILED DESCRIPTION

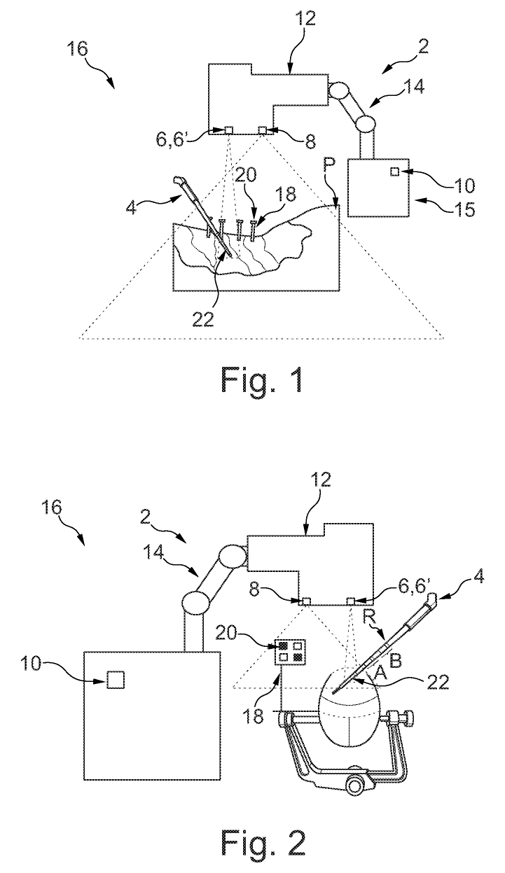

FIG. 1 shows a navigation system 2 for tracking a medical instrument 4 relative to a patient P, comprising a first navigation camera 6 that is set up to capture a current position and/or orientation of the medical instrument 4 in the visible spectrum, a separate second navigation camera 8 that is set up to capture a current position and/or orientation of the patient P in the visible spectrum, and a control unit 10 configured to determine a current relative position and/or orientation of the medical instrument 4 with respect to the patient P from the current position and/or orientation of the medical instrument 4 captured by the first navigation camera 6 and the current position and/or orientation of the patient P captured by the second navigation camera 8.

The navigation system 2 is used here as an example for spinal surgery.

The first navigation camera 6 and the second navigation camera 8 are jointly and firmly attached to each other as an end effector 12 on a robotic arm 14. Robotic arm 14 is attached to a base 15, for example a trolley.

The first navigation camera 6 is a digital surgical microscope 6'.

The second navigation camera 8 is a wide-angle camera. The field of view of the second navigation camera 8 is larger than that of the first navigation camera 6. The fields of view are indicated by dotted lines.

A medical system 16 includes the navigation system 2 and also a patient tracker 18. The patient tracker 18 is designed to be rigidly attached to the patient to reference their position and/or orientation. The patient tracker 18 has an optical pattern 20 in the visible spectrum. FIG. 2 schematically shows the optical pattern 20 of a patient tracker 8.

The second navigation camera 8 is set up to visually capture the optical pattern 20 of the patient tracker 18 and to track the patient P using it.

Here, the medical system 16 has several patient trackers 18, each of which is rigidly attached to a vertebra of the patient P in order to reference or indicate their current position and/or orientation.

The medical system 16 further comprises the medical instrument 4. The medical instrument 4 has an optical pattern 22 in the visible spectrum. The second navigation camera 6 is set up to visually capture the optical pattern 22 of the medical instrument 4 and to track the medical instrument 4 based on this.

FIG. 2 shows navigation system 2 from FIG. 1 as an example during a neural procedure, with patient P lying down. The patient's skull is secured in a head clamp. The patient tracker 20 is rigidly attached to the skull to reference its position and/or orientation.

The optical pattern 22 of the medical instrument 4 is finer and smaller (formed) compared to the optical pattern 18 of the patient tracker 20.

Furthermore, the optical pattern 22 of the medical instrument 4 has redundancies R to make it more robust against partial occlusions. The optical pattern 22 has features, in this case lines, that repeat and redundantly provide the same information (a position and/or orientation of the medical instrument 4). The optical pattern 22 is more robust (against partial occlusions) than the optical pattern 20 of the patient tracker 18, since the patient tracker 18 is less likely to be obscured by, for example, a surgeon or, for example, the patient's blood or anatomical structures, especially since it is used outside the patient and may be removed from the surgical area.

Here, features A and features B of optical pattern 22 provide the same information. If features A of optical pattern 22 are obscured, e.g., by blood during surgery, medical instrument 4 can still be tracked via features B, and vice versa. It is therefore sufficient for the navigation camera 6 to have a clear view of features A or features B. The redundancies are shown schematically here, and the figure is not to scale. It is understood that the redundancies R are arranged on instrument 4 in such a way that they can be seen simultaneously by navigation camera 6.

Furthermore, the optical pattern 22 of the instrument 4 has a lower reflectivity than the optical pattern 20 of the patient tracker 18. Instrument 4 and optical pattern 22 of instrument 4 are sterilizable. The optical pattern 22 is engraved. The Patient Tracker 18 is a disposable product and therefore cannot be sterilized. The optical pattern 20 is glued on.

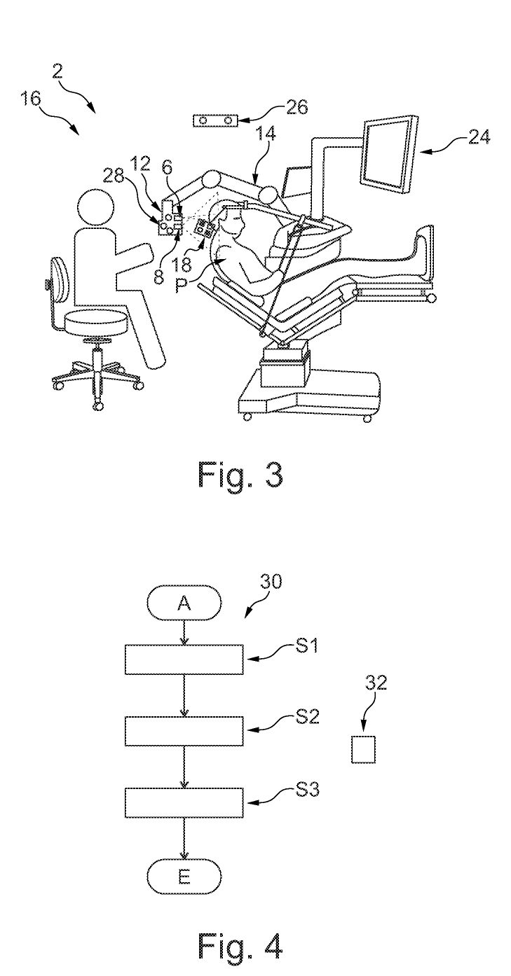

FIG. 3 shows a navigation system 2, similar to that shown in FIGS. 1 and 2, used as an example in a cranial procedure, with the patient P in a semi-sitting position. The navigation system 2 has a display device 24 that is set up to display an image from the first navigation camera 6 and an image from the second navigation camera 8.

Furthermore, FIG. 3 shows a medical system 2 similar to that of FIGS. 1 and 2, wherein the medical system 2 here has a third, separate navigation camera 26 for the infrared (IR) spectrum and at least one IR marker 28 connected to the robotic arm 14, wherein the third navigation camera 26 is configured to track the IR marker 28 in the infrared spectrum. Here, there are three IR markers attached to end effector 12. The patient tracker 18 is rigidly attached to or connected to the patient P via a head clamp.

FIG. 4 shows a navigation method 30. The navigation method 30 is for tracking a medical instrument 4 relative to a patient P and can be performed, for example, by the navigation system 2 and/or the medical system 16 from FIG. 1. It has a start A and an end E and consists of the following steps S:
S1: capturing a current position and/or orientation of the medical instrument 4 in the visible spectrum, preferably via an optical pattern 22 of the medical instrument 4, by a first navigation camera 6;
S2: capturing a current position and/or orientation of the patient P in the visible spectrum, preferably via a patient tracker 18 with an optical pattern 20, by a second, separate navigation camera 8; and
S3: determining a current relative position and/or orientation of the medical instrument 4 with respect to the patient P from the current position and/or orientation of the medical instrument 4 captured by the first navigation camera 6 and the current position and/or orientation of the patient P captured by the second navigation camera 8.

Step S3 can be performed, for example, by a control unit 12.

Furthermore, FIG. 4 shows a computer-readable storage medium 32.

### LIST OF REFERENCE SIGNS

2 Navigation system

4 Medical instrument

P Patient

6 First navigation camera

6‘ Digital surgical microscope

8 Second navigation camera

10 Control unit

12 End effector

14 Robotic arm

15 Base

16 Medical system

18 Patient tracker

20 Optical pattern (of the patient tracker)

22 Optical pattern (of the medical instrument)

R Redundancy

24 Display device

26 Third (IR) navigation camera

28 IR Marker

30 Navigation method

32 Computer-readable storage medium

1. A navigation system for tracking a medical instrument relative to a patient, the navigation system comprising:
a first navigation camera configured to capture a current position and/or orientation of the medical instrument in a visible spectrum;
a second navigation camera configured to capture a current position and/or orientation of the patient in the visible spectrum; and
a control unit configured to determine a current relative position and/or relative orientation of the medical instrument relative to the patient from the current position and/or orientation of the medical instrument captured by the first navigation camera and the current position and/or orientation of the patient captured by the second navigation camera.

2. The navigation system according to claim 1, wherein the first navigation camera and the second navigation camera are mounted together and fixed relative to each other.

3. The navigation system according to claim 2, wherein the first navigation camera and the second navigation camera are mounted as an end effector on a robotic arm.

4. The navigation system according to claim 1, wherein the first navigation camera is a digital surgical microscope.

5. The navigation system according to claim 1, wherein the second navigation camera is a wide-angle camera.

6. The navigation system according to claim 5, wherein the second navigation camera has a larger field of view than the first navigation camera.

7. A medical system comprising:
the navigation system according to claim 1; and
a patient tracker configured to be rigidly attached to the patient to reference the current position and/or orientation of the patient,
the patient tracker having a first optical pattern in the visible spectrum, and
the second navigation camera configured to visually capture the first optical pattern and track the patient via the first optical pattern.

8. The medical system according to claim 7, further comprising a medical instrument, wherein the medical instrument has a second optical pattern in the visible spectrum, and the second navigation camera is configured to visually capture the second optical pattern of the medical instrument and track the medical instrument via the second optical pattern.

9. The medical system according to claim 8, wherein the second optical pattern of the medical instrument is finer and/or smaller than the first optical pattern of the patient tracker.

10. The medical system according to claim 8, wherein the second optical pattern of the medical instrument has a lower reflectivity than the first optical pattern of the patient tracker.

11. The medical system according to claim 8, wherein the second optical pattern of the medical instrument is engraved and/or coated for reusability and sterilizability.

12. The medical system according to claim 11, wherein the patient tracker is a disposable product.

13. The medical system according to claim 11, wherein the first optical pattern of the patient tracker is not sterilizable.

14. The medical system according to claim 8, wherein the second optical pattern of the medical instrument is more robust against partial occlusion than the first optical pattern of the patient tracker.

15. The medical system according to claim 7, further comprising:
a robotic arm;
a third navigation camera for an infrared spectrum; and
at least one IR marker connected to the robotic arm,
wherein the third navigation camera is configured to track the at least one IR marker in the infrared spectrum.

16. A navigation method for tracking a medical instrument relative to a patient, the navigation method comprising the steps of:
capturing a current position and/or orientation of the medical instrument in a visible spectrum by a first navigation camera;
capturing a current position and/or orientation of the patient in the visible spectrum by a second navigation camera; and
determining a current relative position and/or orientation of the medical instrument with respect to the patient from the current position and/or orientation of the medical instrument captured by the first navigation camera and the current position and/or orientation of the patient captured by the second navigation camera.

17. The navigation method according to claim 16, wherein the step of capturing the current position and/or orientation of the medical instrument in the visible spectrum comprises visually capturing a first optical pattern of the medical instrument.

18. The navigation method according to claim 16, wherein the step of capturing the current position and/or orientation of the patient in the visible spectrum comprises visually capturing a patient tracker with a second optical pattern.
